(19) United States
(12) Patent Application Publication
(10) Pub. No.: US 2026/0345838 A1
(43) Pub. Date: Nov. 5, 2026

(54) IDENTIFICATION AND/OR TREATMENT OF CANCER

(71) Applicant: CLARITY PHARMACEUTICALS LIMITED, Eveleigh, New South Wales (AU)

(72) Inventors: Eva LENGYELOVA, Eveleigh (AU); Ellen Marianne VAN DAM, Eveleigh (AU); Robert MILLER, Eveleigh (AU)

(21) Appl. No.: 19/124,367

(22) PCT Filed: Oct. 26, 2023
(86) PCT No.: PCT/AU2023/051077

§ 371 Date: Apr. 25, 2025

(30) Foreign Application Priority Data
Oct. 26, 2022 (AU) 2022903169

(51) Int. Cl. A61K 51/08; C12Q 1/6886; G01N 33/534; G01N 33/57555

(52) U.S. Cl. CPC A61K 51/088; C12Q 1/6886; G01N 33/534; G01N 33/57555; C12Q 2600/106; C12Q 2600/158; G01N 2333/726; G01N 2800/52; G01N 2800/7028

(57) ABSTRACT

The present invention relates generally to the identification and/or treatment of cancer and in particular cancers in which gastrin releasing peptide receptor (GRPR) is expressed. In particular embodiments, the present invention relates to the identification and/or treatment of prostate cancer and in particular GRPR expressing metastatic castrate resistance prostate cancer.

### FIELD

The present invention relates generally to the identification and/or treatment of cancer and in particular cancers in which gastrin releasing peptide receptor (GRPR) is expressed. In particular embodiments, the present invention relates to the identification and/or treatment of prostate cancer and in particular GRPR expressing metastatic castrate resistance prostate cancer.

### BACKGROUND

Prostate cancer (PCa) is the second most frequent malignancy in men worldwide. The American Cancer Society estimates 268,490 new cases of PCa will be diagnosed in the United States of America (USA) in 2022, accounting for approximately 27% of all new estimated cancer cases in men and approximately 14% of all new estimated cancer case. The incidence of PCa correlates with age, running between 30% for patients 40-50 years old to 50-80% for patients aged 80 and over. The median age of patients diagnosed with PCa is 67 years and the median age at death is 81 years. The etiology of the disease is unclear; the main risk factors include advanced age, ethnicity, and positive family history. Many patients with PCa have indolent disease, where the patient's serum prostate-specific antigen (PSA) values are followed without further treatment. At initial presentation, 74% of patients have local disease, 13% have regional disease, and 7% have metastatic disease, with the remaining 6% classified as unknown. Since 2000, 5-year relative survival rate for local or regional PCa has been over 97%. However, for metastatic (distant) PCa, the 5-year survival rate drops dramatically to 30%. Between 20-40% of patients will experience rising PSA levels (biochemical recurrence or failure) within 10 years of primary PCa treatment. Approximately 25-35% of patients with recurrent disease have locally recurrent disease, 20-25% have metastatic disease, and 45-55% will have both.

Gastrin Releasing Peptide Receptor (GRPR) is a transmembrane G-protein coupled receptor that has various physiological functions in the gastrointestinal tract and nervous system. Its pharmacological activities, through binding of its ligand Gastrin Releasing Peptide (GRP), include the stimulation of hormone releasing, like gastrin and somatostatin, as well as stomach and intestine smooth muscle contraction. Gastrin Releasing Peptide Receptor expression is upregulated in many human cancers, including PCa, breast cancer, glioma, ovarian cancer, lung cancer, and gastrinoma and gastrointestinal stromal tumours [GIST]), with its normal biodistribution mainly concentrated in the pancreas and gastrointestinal tract. Although the correlation between GRPR expression and clinical features in PCa, such as Gleason score, stage of disease and PSA levels, have been evaluated, the results remain inconclusive. Indeed, a recent study showed uptake of a gallium-68-labeled GRPR targeting agent (⁶⁸Ga-RM2) across all PSA levels in biochemical recurrent PCa.

Several GRPR-targeting imaging and theranostic agents are under clinical investigation, but none are widely available or have been approved by regulatory authorities to date. Expression of GRPR in PCa has been reported, with expression found in 75-100% of samples analysed. Furthermore, clinical studies using GRPR-targeting imaging agents such as ⁶⁸Ga-RM2, ⁶⁸Ga-NeoB, or ⁶⁸Ga-SB3 reported high uptake in primary PCa lesions as well as metastases, and a positivity/detection rate ranging from 31% to 100% as measured in several studies. No clinical therapy studies targeting GRPR have been reported to date. However, a first in human dosimetry study of ¹⁷⁷Lu-RM2 in patients with metastatic castrate-resistant prostate cancer (mCRPC) who were ineligible for ¹⁷⁷Lu-PSMA-617 therapy showed that 4.5 GBq of ¹⁷⁷Lu-RM2 was well tolerated by all 4 patients and no side effects were observed. Therapeutically relevant absorbed doses to tumor were recorded while rapid clearance from normal organs was seen.

A need exists to improve patient outcomes for cancers in which gastrin releasing peptide receptor (GRPR) is expressed including prostate cancer patients and especially those diagnosed with mCRPC who are ineligible for ¹⁷⁷Lu-PSMA-617 therapy based on their prostate specific membrane antigen (PSMA) expression.

### SUMMARY

The present inventors have been developing a radiolabelled antagonist of GRPR for the diagnosis and treatment of prostate cancer. The product is copper-complexed MeCOSar-PEG₄-D-Phe-Gln-Trp-Ala-Val-Gly-His-Sta-Leu-NH₂ (Cu-SAR-BBN). Cu-SAR-BBN uses a radioactive form (radionuclide) of copper, copper-64 (⁶⁴Cu) to image cancers using positron emission tomography (PET) and then copper-67 (⁶⁷Cu) for therapy by internal beta-radiation. This ‘theranostic’ approach is well established in neuroendocrine tumors with the use of the approved products: NETSPOT® or Detectnet™ for imaging and LUTATHERA® for therapy. These products consist of DOTA-octreotate, which targets somatostatin receptor-positive lesions but use different radionuclides (gallium-68 [⁶⁸Ga] or ⁶⁴Cu for imaging and lutetium-177 [¹⁷⁷Lu] for therapy). Similarly, the prostate specific membrane antigen (PSMA) targeting agents Locametz® (⁶⁸Ga-PSMA-11) and Pluvicto™ (¹⁷⁷Lu-PSMA-617) were recently approved by the USA-FDA as an imaging agent for patient selection and therapeutic agent for metastatic castrate resistant prostate cancer (mCRPC), respectively. Targeted radionuclide therapy achieves anti-tumor effects because higher doses of radiation are administered than for diagnostic purposes and the radionuclides used for therapy that impart much greater energy to cellular structures such as deoxyribonucleic acid (DNA), resulting in target cell cytotoxicity. Effectiveness of this therapy depends on delivering the highest possible radiation dose to the tumor while sparing normal organs and tissues from damage. This is achieved by highly specific, high-affinity binding to the receptors that are overexpressed on the tumors. Multiple GRPR-targeted radiopharmaceuticals for therapy are disclosed herein. Until now no clinical studies using ⁶⁷Cu labelled products have been reported to date. The inventors have identified that ⁶⁷Cu has similar characteristics as ¹⁷⁷Lu; both emit beta minus particles with similar maximum energy (577 keV³⁰ and 498 keV³¹, respectively) and therefore potential similar range in tissue. In addition, preclinical efficacy studies in tumor bearing mice showed ⁶⁷Cu-tracers to be as efficacious as ¹⁷⁷Lu-tracers.

Accordingly, a first aspect the invention provides a method of treating a patient diagnosed with GRPR expressing cancer, said method including the step of administering an effective amount of a compound of Formula (I) or a pharmaceutically acceptable salt thereof complexed to a ⁶⁷Cu radioisotope:

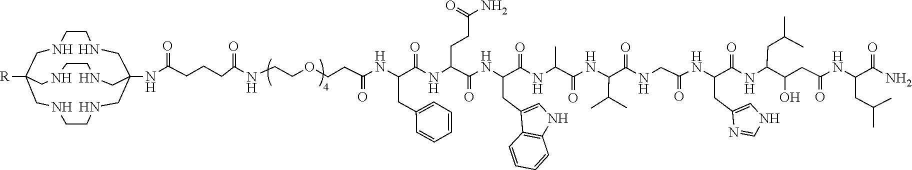

where R is CH₃C(O)—;

(⁶⁷Cu-SAR-BBN)

and
wherein the dose of radiation delivered by the radioisotope is sufficient to reduce the size of one or more lesions associated with said cancer.

In certain embodiments the GRPR expressing cancer is selected from prostate cancer, breast cancer, glioma, ovarian cancer, lung cancer, and gastrinoma and gastrointestinal stromal tumours [GIST]).

In a second aspect the invention provides a method of treating a patient diagnosed with GRPR expressing metastatic castrate resistance prostate cancer, said method including the step of administering an effective amount of a compound of Formula (I) or a pharmaceutically acceptable salt thereof complexed to a ⁶⁷Cu radioisotope:

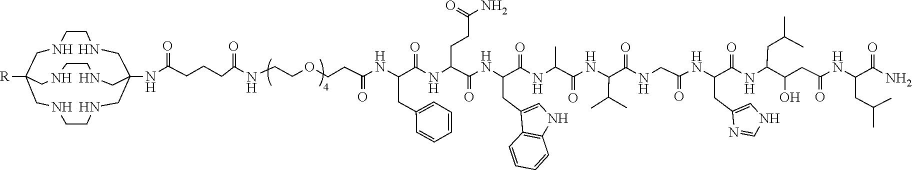

where R is CH₃C(O)—;

(⁶⁷Cu-SAR-BBN)

and
wherein the dose of radiation delivered by the radioisotope is sufficient to reduce the size of one or more lesions associated with said cancer.

In a third aspect the invention also provides a method of treating a patient diagnosed with GRPR expressing metastatic castrate resistance prostate cancer who is ineligible for therapy with ¹⁷⁷Lu-PSMA-617, said method including the step of administering an effective amount of a compound of Formula (I) or a pharmaceutically acceptable salt thereof complexed to a ⁶⁷Cu radioisotope:

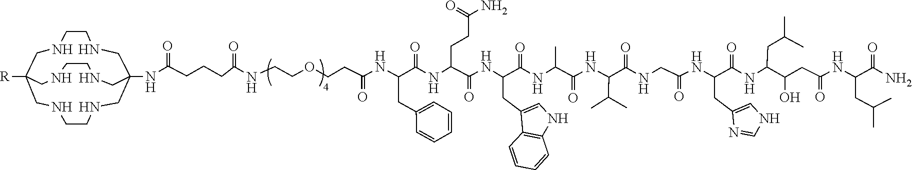

where R is CH₃C(O)—;

(⁶⁷Cu-SAR-BBN)

and
wherein the dose of radiation delivered by the radioisotope is sufficient to reduce the size of one or more lesions associated with said cancer.

In a fourth aspect the invention provides a method of identifying and treating a patient diagnosed with GRPR expressing metastatic castrate resistance prostate cancer, said method including the steps of:
(i) administering an effective amount of a compound of Formula (I) or a pharmaceutically acceptable salt thereof complexed to a ⁶⁴Cu radioisotope:

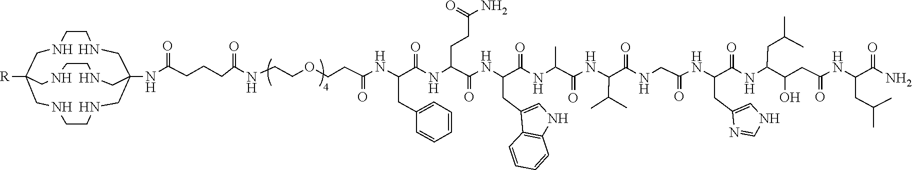

where R is CH₃C(O)—;

(⁶⁴Cu-SAR-BBN)

and
wherein the dose of radiation delivered by the ⁶⁴Cu radioisotope is sufficient to identify one or more lesions associated with said cancer; and
(ii) administering an effective amount of a compound of Formula (I) or a pharmaceutically acceptable salt thereof complexed to a ⁶⁷Cu radioisotope:

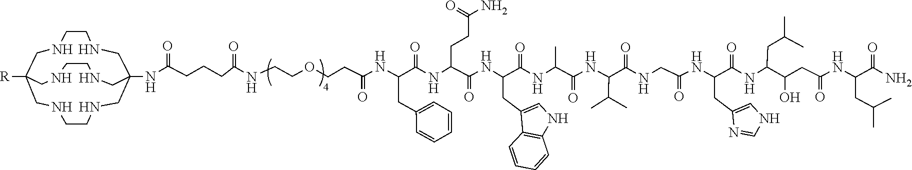

where R is CH₃C(O)—;

(⁶⁷Cu-SAR-BBN)

and
wherein the dose of radiation delivered by the ⁶⁷Cu radioisotope is sufficient to reduce the size of said one or more lesions associated with said cancer.

In a fifth aspect the invention provides a method of identifying and treating a patient diagnosed with GRPR expressing metastatic castrate resistance prostate cancer who is ineligible for therapy with ¹⁷⁷Lu-PSMA-617, said method including the steps of:
(i) administering an effective amount of a compound of Formula (I) or a pharmaceutically acceptable salt thereof complexed to a ⁶⁴Cu radioisotope:

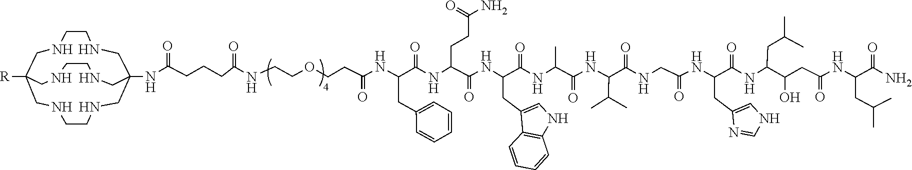

where R is CH₃C(O)—;

(⁶⁴Cu-SAR-BBN)

and
wherein the dose of radiation delivered by the ⁶⁴Cu radioisotope is sufficient to identify one or more lesions associated with said cancer; and
(ii) administering an effective amount of a compound of Formula (I) or a pharmaceutically acceptable salt thereof complexed to a ⁶⁷Cu radioisotope:

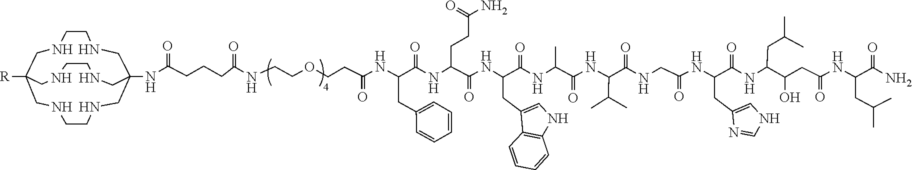

where R is CH₃C(O)—;

(⁶⁷Cu-SAR-BBN)

and
wherein the dose of radiation delivered by the ⁶⁷Cu radioisotope is sufficient to reduce the size of said one or more lesions associated with said cancer.

In certain embodiments and with reference to all aspects, the stereochemistry of the peptide portion of the compound of Formula (I) is depicted as follows:

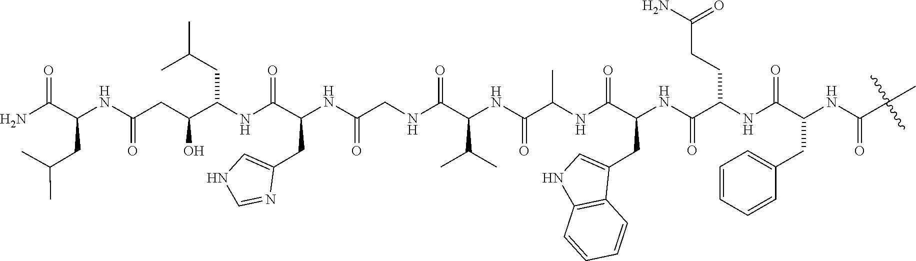

In some embodiments of the present invention, the dose of radiation delivered by the ⁶⁴Cu radioisotope to identify one or more lesions is between about 100 MBq and about 300 MBq. In certain embodiments, the dose of radiation delivered by the ⁶⁴Cu radioisotope to identify one or more lesions is about 100 MBq, about 120 MBq, about 140 MBq, about 160 MBq, about 180 MBq, about 200 MBq, about 220 MBq, about 240 MBq, about 260 MBq, about 280 MBq or about 300 MBq.

In certain embodiments of the present invention, the dose of radiation provided by the ⁶⁷Cu radioisotope to reduce the size of one or more lesions associated with the cancer is about 4 GBq, about 6 GBq, about 8 GBq, about 10 GBq, about 12 GBq, about 14 GBq, about 16 GBq, about 18 GBq, about 20 GBq, about 22 GBq or about 24 GBq.

In certain embodiments of the methods for treatment of a cancer as disclosed herein, the compound of Formula (I) complexed with a ⁶⁷Cu radioisotope is administered once. In other embodiments, the compound of Formula (I) complexed with a ⁶⁷Cu radioisotope is administered to the same subject more than once. In other embodiments, the compound of Formula (I) complexed with a ⁶⁷Cu radioisotope is administered two times, three times, four times or five times to the same subject.

As discussed above, the compound of Formula (I) targets the gastrin releasing peptide receptor (GRPR), which is associated with various cancer types. In certain embodiments, the cancer is a breast cancer. In other embodiments, the cancer is a particular subset or type of breast cancer. In some embodiments, the breast cancer is associated with the expression of one or more of oestrogen, progesterone or HER2 receptors.

Different subtypes of breast cancer show varying levels of sensitivity towards conventional ¹⁸FDG-PET imaging, which means that the use of ¹⁸FDG-PET imaging may not necessarily identify all breast cancer lesions. For example, ¹⁸FDG has lower sensitivity for ER+/PR+ breast cancer, when compared to triple negative (i.e. ER/PR/HER2 negative) subtypes. As seen in FIGS. 1 to 6, subjects showing clinical progression of metastatic ER+/PR+/HER2− were imaged by ¹⁸FDG-PET and ⁶⁴Cu-Sar-BBN. Compared to conventional imaging with FDG, combined patient analysis of images taken after administration ⁶⁴Cu-Sar-BBN showed higher mean total tumor volume, SUVₘₐₓ and total number of lesions. This suggests that the use of ⁶⁴Cu-Sar-BBN may provide a more comprehensive evaluation of lesions in a subject. For example, FIG. 2 shows a subject with classical lobular breast cancer with extensive metastases throughout, with a calculated mean total tumor volume that is four times higher than the tumor volume calculated by imaging with ¹⁸FDG. In FIG. 4, conventional imaging by FDG identified less than 3 lesions, however imaging of the same subject after administration of ⁶⁴Cu-Sar-BBN identified at least 19 lesions.

In a further aspect, the present invention provides a method for predicting the response of a patient to treatment of a cancer with a compound of Formula (I) complexed with ⁶⁷Cu, the method comprising detecting and enumerating circulating tumor DNA (ctDNA) associated with one or more genes in the patient and correlating the amount of ctDNA detected with the response of the patient to said treatment, wherein the cancer is a GRPR expressing cancer and wherein the one or more genes is selected from the group consisting of BRCA1, BRCA2, RB and p53.

The methods for predicting the response of a patient to treatment with ⁶⁷Cu means that a patient may not be administered the compound of Formula (I)⁶⁷Cu, especially where the ctDNA detected in the patient indicates that the response elicited does not result in the reduction in the size of one or more lesions of the cancer associated with the group.

### BRIEF DESCRIPTION OF THE FIGURES

FIG. 1. A patient with clinical progression of metastatic ER+/PR+/HER2− invasive ductal breast cancer requiring re-staging was imaged with ¹⁸FDG-PET (A). The same patient was administered 200 MBq of ⁶⁴Cu-Sar-BBN and imaged by PET (B).

FIG. 2. A patient with clinical progression of metastatic ER+/PR+/HER2− classical lobular breast cancer requiring re-staging was imaged with ¹⁸FDG-PET (A). The same patient was administered 200 MBq of ⁶⁴Cu-Sar-BBN and imaged by PET (B). Biopsy of the patient showed metastases in the liver. Imaging by PET after administration of ⁶⁴Cu-Sar-BBN showed a higher SUVₘₐₓ than imaging by conventional FDG means. The total tumor volume detected by ⁶⁴Cu-Sar-BBN was approximately four times higher than with FDG imaging, which strongly suggests that identification of cancerous lesions can be achieved by ⁶⁴Cu-Sar-BBN. This Figure also shows that lesions that were not detected by conventional FDG imaging could be located with ⁶⁴Cu-Sar-BBN

FIG. 3. A patient with clinical progression of metastatic ER+/PR+/HER2− invasive ductal breast cancer requiring re-staging was imaged with ¹⁸FDG-PET (A). The same patient was administered 200 MBq of ⁶⁴Cu-Sar-BBN and imaged by PET (B). Biopsy of the patient showed metastases in the liver. Imaging with ⁶⁴Cu-Sar-BBN revealed lesions that were not detected by conventional FDG imaging.

FIG. 4. A patient with clinical progression of metastatic ER+/PR+/HER2− invasive ductal breast cancer requiring re-staging was imaged with ¹⁸FDG-PET (A). The same patient was administered 200 MBq of ⁶⁴Cu-Sar-BBN and imaged by PET (B). Biopsy of the patient showed metastases in the chest wall. The total tumor volume detected by ⁶⁴Cu-Sar-BBN was higher than with FDG imaging, which strongly suggests that identification of cancerous lesions can be achieved by ⁶⁴Cu-Sar-BBN.

FIG. 5. A patient with clinical progression of metastatic ER+/PR+/HER2− classical lobular breast cancer requiring re-staging was imaged with ¹⁸FDG-PET (A). The same patient was administered 200 MBq of ⁶⁴Cu-Sar-BBN and imaged by PET (B). Maximum intensity projections and axial slices of the patient after imaging with ¹⁸FDG-PET (C) and ⁶⁴Cu-Sar-BBN PET (D) are also provided. Biopsy of the patient showed metastases in the skin. Imaging by ⁶⁴Cu-Sar-BBN did identified various tumors throughout the patient, whereas conventional imaging by FDG did not reveal any tumors. This also strongly suggests that identification of cancerous lesions can be achieved by ⁶⁴Cu-Sar-BBN.

FIG. 6. A patient with clinical progression of metastatic ER+/PR+/HER2− invasive ductal breast cancer requiring re-staging was imaged with ¹⁸FDG-PET (A). The same patient was administered 200 MBq of ⁶⁴Cu-Sar-BBN and imaged by PET (B). Biopsy of the patient showed metastases in the pleura. Imaging with ⁶⁴Cu-Sar-BBN revealed lesions that were not detected by conventional FDG imaging.

FIG. 7. A post-radical prostatectomy prostate cancer patient showed serial negative PSMA-PET, as determined with ⁶⁸Ga-PSMA-11 imaging (A). Other imaging modalities, such as bone scan (B) and ¹⁸FDG-PET (C) also failed to identify any recurrent lesions. The patient was administered 200 MBq of ⁶⁴Cu-Sar-BBN and the resultant PET images (D and E) revealed the presence of additional lesions. Imaging with ⁶⁴Cu-Sar-BBN revealed lesions that were not detected by conventional imaging modalities.

FIG. 8. A post-radical prostatectomy prostate cancer patient showed serial negative PSMA-PET, as determined with ⁶⁸Ga-PSMA-11 imaging (A). Other imaging modalities, such as bone scan (B) also failed to identify any recurrent lesions. The patient was administered 200 MBq of ⁶⁴Cu-Sar-BBN and the resultant PET images (C and D) revealed the presence of additional lesions in the prostate.

FIG. 9. A post-radical prostatectomy prostate cancer patient showed serial negative PSMA-PET, as determined with ⁶⁸Ga-PSMA-11 imaging (A). The patient was administered 200 MBq of ⁶⁴Cu-Sar-BBN and the resultant PET images (B and C) revealed the presence of additional lesions in the prostate that were not detected by conventional imaging modalities.

FIG. 10. A post-radical prostatectomy prostate cancer patient showed serial negative PSMA-PET, as determined with ⁶⁸Ga-PSMA-11 imaging (A and B). The patient was administered 200 MBq of ⁶⁴Cu-Sar-BBN and the resultant PET images (C) revealed the presence of additional lesions that were not detected by conventional imaging modalities.

### DETAILED DESCRIPTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “about” or “approximately” as used herein means within an acceptable error range for the particular value as determined by one of ordinary skill in the art, which will depend in part on how the value is measured or determined, i.e., the limitations of the measurement system.

Unless defined otherwise, all technical and scientific terms used herein have the same meaning as commonly understood by those of ordinary skill in the art to which the invention belongs. For the purposes of the present invention, the following terms are defined below.

As used herein reference to ¹⁷⁷Lu-PSMA-617 refers to the following compound:

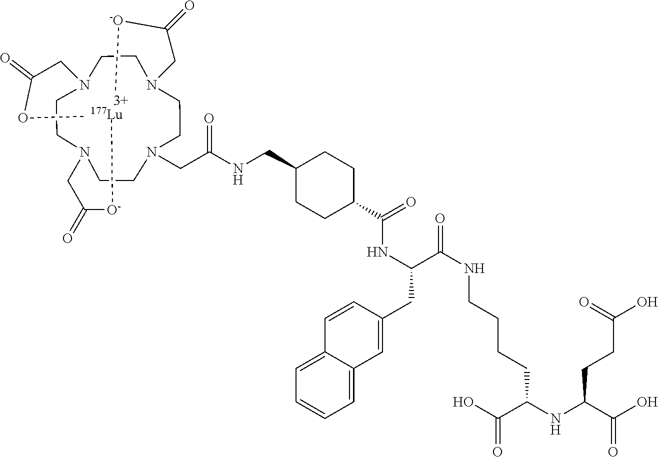

As used herein in certain embodiments the patient is ineligible for ¹⁷⁷Lu-PSMA-617 therapy. As used herein the skilled person may be able to determine such patient groups of based on the following criteria: uptake of ⁶⁸Ga-PSMA-11 or ¹⁸F-DCFPyL in one or more lesions is negative (equal to or lower than that of liver parenchyma), or any one lesion larger than the size criteria is negative [size criteria: organs ≥1 cm, lymph nodes ≥2.5 cm, bones (soft tissue component) ≥1 cm].

### ⁶⁸Ga-PSMA-11 Being:

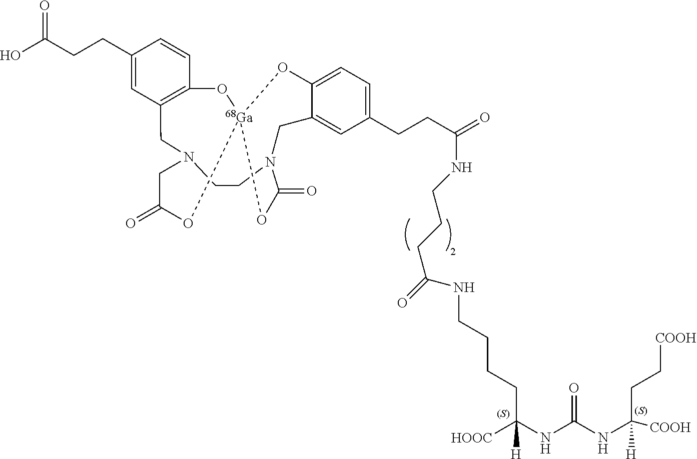

### ¹⁸F-DCFPyL Being:

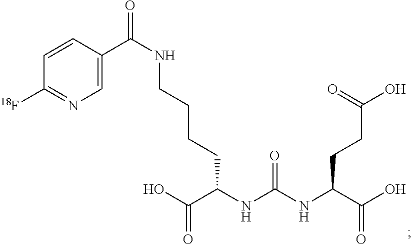 [SMILES: O=C(O)CC[C@H](NC(=O)N[C@@H](CCCCNC(=O)c1ccc([18F])nc1)C(=O)O)C(=O)O]

In some embodiments, the dose of radiation delivered by the ⁶⁷Cu radioisotope is between about 6 GBq and about 56 GBq. In some embodiments, the dose of radiation delivered by the ⁶⁷Cu radioisotope is about 6 GBq, about 10 GBq or about 14 GBq. In some embodiments, the dose of radiation delivered by the ⁶⁷Cu radioisotope is more than about 14 GBq. In some embodiments, the dose of radiation delivered by the ⁶⁷Cu radioisotope is about 20 GBq, about 25 GBq, about 30 GBq, about 35 GBq, about 40 GBq, about 45 GBq, about 50 GBq or about 55 GBq. In other embodiments, the dose of radiation delivered by the ⁶⁷Cu radioisotope is the highest dose that is tolerated by the subject.

In some embodiments the prostate cancer is a GRPR expressing metastatic castrate resistance prostate cancer (GRPRmCRPC), and in certain embodiments is a progressive GRPRmCRPC despite prior androgen deprivation therapy and at least either enzalutamide and/or abiraterone (or other such androgen receptor pathway inhibitors).

In a further embodiment the patient is a male subject with a castrate level of serum/plasma testosterone of about <50 ng/dL or about <1.7 nmol/L.

In a further embodiment the patient is a male subject with a prostate specific antigen (PSA) value of 50 or above for more than 3 weeks before administering a compound of Formula (I) or a pharmaceutically acceptable salt thereof complexed to a ⁶⁷Cu radioisotope.

In a further embodiment the patient experiences a decrease in percentage of PSA and/or, alkaline phosphatase (ALP) and/or, lactate dehydrogenase (LDA) biomarkers after one, two, three or four treatment administration cycles of a compound of Formula (I) or a pharmaceutically acceptable salt thereof complexed to a ⁶⁷Cu radioisotope relative to a baseline taken prior to each the treatment.

In a further embodiment the method comprises administering to a subject an effective amount of a compound of Formula (I) or a pharmaceutically acceptable salt thereof complexed to a ⁶⁷Cu radioisotope with 1, 2, 3, or 4 treatment cycles.

In further embodiments the method comprises administering to a subject an aqueous formulation of a compound of Formula (I) or a pharmaceutically acceptable salt thereof complexed to a ⁶⁷Cu radioisotope, at least 2 administrations, about 6 to 14 weeks apart, of a dose to provide a 6-14 GBq level by IV slow infusion over about 30 minutes to about 60 minutes. In other embodiments, the method comprises at least 3 administrations to the subject. In other embodiments, the method comprises at least 4 administrations to the subject.

In an embodiment, the method further comprises radioimaging the subject by PET, SPECT and/or CT, preferably after each treatment cycle, and preferably with ⁶⁴Cu-SAR-BBN by PET.

In an embodiment, a positive PET and/or CT scan is based on a visualisation of ⁶⁴Cu-SAR-BBN (formula (I)) PET/CT scan, where ⁶⁴Cu-SAR-BBN uptake (standardized uptake value [SUV] max) of at least 1 known lesion is higher than that of the gastrointestinal tract on the 1 hour positron emission tomography (PET)/computed tomography (CT) scan.

The present inventors believe that the images of the subject obtained by PET and/or CT after administration of the ⁶⁴Cu-complexed compound of Formula (I) in accordance with the above aspects may dictate or at least assist with the corresponding dose of the ⁶⁷Cu-complexed compound of Formula (I) that is used for the treatment of the GRPR expressing metastatic castrate resistance prostate cancer. Accordingly, one of the benefits of the present invention is that the same BBN-linked compound may be used in a complete diagnosis (identification)-therapy regime by replacing the Cu radioisotope from ⁶⁴Cu to ⁶⁷Cu.

Thus the present inventors also believe that administration of more than one dose (ie multiple treatment cycles) of the compounds and formulations described herein for the treatment of GRPR expressing metastatic castrate resistance prostate cancer leads to greater accumulation of the radioisotope at the target site. Without wishing to be bound by theory, the present inventors believe that the use of the radiolabelled compounds described herein allow for greater doses of radiation to be delivered without an increase in the expected adverse effects. This therefore leads to greater efficacy in treatment. The before herein disclosed diagnosis (identification) methods can be used before, or between treatment cycles in order to assess the effectiveness of the treatment.

Without wishing to be bound by theory, the present inventors believe that administration of more than one administration dose of the formulations described herein for the treatment of GRPR expressing metastatic castrate resistance prostate cancer leads to higher absorbed radiation doses at the cancer site, which leads to greater efficacy of treatment. This means that repeat administrations of the formulation containing the compound of Formula (I) complexed with a radioisotope may lead to greater survival of the subject, when compared with single administrations of the formulations as disclosed herein. In an embodiment, the method comprises the sequential administration of more than one dose of the compound described in the first and second aspects. In some embodiments, the sequential doses are administered between about 3 weeks, about 4 weeks, about 5 weeks, about 6 weeks, about 7 weeks about 8 weeks, about 9 weeks, about 10 weeks, about 11 weeks, about 12 weeks, about 13 weeks, about 14 weeks, about 15 weeks or about 16 weeks apart (such as between about 4 to 14 weeks apart). In an embodiment, the sequential doses are administered about 6 weeks apart. In an embodiment, the total dose of radiation delivered to the bone marrow of the subject is less than about 2 Gy. In another embodiment, the total dose of radiation delivered to the kidneys of the subject is less than about 23 Gy.

The treatment methods of aspects one and two may comprise administration of multiple doses of the ⁶⁷Cu complex containing the compound of Formula (I) and the radioisotope, where the doses administered are the same or different. In some embodiments, where multiple doses are administered, the second and any subsequent doses may be higher than the original dose. In some embodiments, multiple doses are administered where the doses are the same. In another embodiment, multiple doses are administered where the doses are different. One skilled in the art would understand that since methods discussed herein incorporate the use of a radioisotope, there is a maximum total dose of radiation that a subject may be given. In some embodiments, multiple doses are administered until the cumulative dose of radiation delivered to the kidneys of the subject reaches about 23 Gy. In some embodiments, multiple doses are administered, until the cumulative dose of radiation delivered to the bone marrow of the subject reaches about 2 Gy.

In certain embodiments, the ^{64/67}Cu complexed compounds of formula (I) are administered as an aqueous formulation intended to be administered intravenously (IV). In another embodiment, the aqueous formulation is administered by slow infusion. In a preferred embodiment, the aqueous formulation is administered intravenously by slow infusion, for instance between about 30-60 minutes.

In certain embodiments the ⁶⁴Cu complexed compounds of formula (I) are administered as an aqueous formulation intended to be administered intravenously (IV) slow bolus injection.

In certain embodiments the ⁶⁷Cu complexed compounds of formula (I) are administered as an aqueous formulation intended to be administered intravenously (IV) slow infusion over about 30 minutes-60 minutes, and preferably 30 minutes.

The methods disclosed herein include the administration of a radioisotope that emits ionising radiation. Since the kidneys are responsible for blood filtration, the kidneys of a subject to which the formulations comprising the compound of Formula (I) and a radioisotope has been administered are at risk of absorbing unwanted radiation as a result of active reabsorption and retention of the radiolabelled compound of Formula (I). Prevention of nephrotoxicity may be achieved by co-administration of cationic amino acids that competitively inhibit the reabsorption of the compound of Formula (I) and thus the radioisotope. In some embodiments, the method of the second aspect further comprises the administration of a formulation containing one or more amino acids, or salts thereof. In some embodiments, one or more amino acids are in a cationic form. In some embodiments, the formulation containing one or more amino acids comprises lysine, or a salt thereof. In other embodiments, the formulation containing one or more amino acids comprises arginine, or a salt thereof. In a preferred embodiment, the method comprises the administration of a formulation comprising lysine and arginine, or salts thereof.

The term “pharmaceutically acceptable salts” refers to salts that retain the desired biological activity of the above-identified compounds, and include pharmaceutically acceptable acid addition salts and base addition salts. Suitable pharmaceutically acceptable acid addition salts of compounds of Formula (I) may be prepared from an inorganic acid or from an organic acid. Examples of such inorganic acids are hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic acids. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of a compound with the appropriate inorganic or organic acid via any of a number of known methods. Alternatively, alkali and alkaline earth metal salts can be prepared by reacting a compound with the appropriate base via a variety of known methods. The following are further examples of acid salts that can be obtained by reaction with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. Additional information on pharmaceutically acceptable salts can be found in Remington's Pharmaceutical Sciences, 19th Edition, Mack Publishing Co., Easton, PA 1995. In the case of agents that are solids, it is understood by those skilled in the art that the inventive compounds, agents and salts may exist in different crystalline or polymorphic forms, all of which are intended to be within the scope of the present invention and specified formulae.

The compound formulations of this invention for injection comprise pharmaceutically acceptable sterile aqueous solutions. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. The formulations may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminium monostearate and gelatin. The injectable formulations can be sterilized, for example, by filtration through a bacterial-retaining filter, or by incorporating sterilizing agents in the form of sterile solid compositions that can be dissolved or dispersed in sterile water or other sterile injectable medium just prior to use. The pharmaceutical formulation may further comprise a pH controller. Examples of suitable pH controllers include hydrochloric acid, sodium hydroxide and the like. Identification of preferred pH ranges (where appropriate) and suitable excipients is routine in the art, for example, as described in Katdare and Chaubel (2006) Excipient Development for Pharmaceutical, Biotechnology and Drug Delivery Systems (CRC Press).

The formulations of the invention as disclosed herein may be provided in a pharmaceutically acceptable carrier or diluent. As will be appreciated by those skilled in the art, the choice of pharmaceutically acceptable carrier or diluent will be dependent on the route of administration and on the nature of the condition and subject to be treated. The particular carrier or diluent and route of administration may be readily determined by a person skilled in the art. The carrier or diluent and route of administration should be carefully selected so as to ensure activity of the compound of Formula (I) upon arrival at the site of action.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination. For injection, compositions of the invention may be formulated in aqueous solutions, suitably in physiologically compatible buffers such as Hanks' solution, Ringer's solution or physiological saline buffer.

For the treatment of GRPR expressing metastatic castrate resistance prostate cancer the compound of Formula (I) is complexed with a ⁶⁷Cu-radioisotope. The present inventors have found that the sarcophagine fragment of Formula (I) has a strong affinity for copper isotopes and is capable of complexing and retaining a radioisotope for a time that is sufficient for the purposes of treatment, even after administration to a subject. The half-life of the ⁶⁷Cu radioisotope is approximately 60 hours and undergoes beta decay, thus making the isotope suitable for localised radiotherapy. Since decay of the ⁶⁷Cu radioisotope is accompanied by gamma radiation, the treatment of a subject to which the compound of Formula (I) complexed with ⁶⁷Cu may be monitored and imaged by single-photon emission computed tomography (SPECT). In an embodiment, the method for treating a subject in need thereof by administration of a compound of Formula (I) complexed with ⁶⁷Cu includes monitoring and/or imaging by SPECT. Other imaging techniques during treatment may also be used, for example, MRI and CT. In a preferred embodiment, the method for treatment includes imaging by SPECT and/or CT.

For the purposes of radioimaging, the dose of radiation delivered should be sufficient to provide images of sufficient quality without administering an excess amount to the patient. The dose of radiation (and subsequently the amount of the radiolabelled compound of Formula (I) complexed with a ⁶⁴Cu radioisotope) to be administered for the purposes of radioimaging may be determined based on the bodyweight of the subject. For the purposes of treatment, the dose of radiation to be administered and delivered to the subject by a ⁶⁷Cu radioisotope may be determined based on both bodyweight of the subject and the quality of the images obtained via radioimaging after administration of the compound of Formula (I) complexed with a ⁶⁴Cu radioisotope. In some embodiments, the radiolabelled compound of Formula (I) complexed with a ⁶⁴Cu radioisotope is used to model the distribution of the corresponding compound of Formula (I) complexed with a ⁶⁷Cu radioisotope.

The units of radioactivity as recited herein are given in gray (Gy) or becquerel (Bq). It will be appreciated that the dose of radiation may be converted from one unit to another using known conversion factors and that other units for the amount of radioactivity that are not explicitly recited herein may also be used.

As used herein the terms “treating”, or “treatment” and grammatical equivalents refer to any and all uses which remedy the stated cancer, prevent, retard or delay the establishment of the disease, or otherwise prevent, hinder, retard, or reverse the progression of the disease. Thus the term “treating” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

As used herein, the term “cancer” broadly encompasses neoplastic diseases characterised by abnormal cell growth with the potential to invade or spread to other parts of the body. The cancer may be benign, which does not spread to other parts of the body. The cancer may be malignant, meaning that the cancer cells can spread through the circulatory system or lymphatic system. The term as used herein includes all malignant, i.e. cancerous, disease states. The cancer may be present as a tumour.

As used herein, the term “tumour” refers to any malignant cancerous or pre-cancerous cell growths. The term is particularly directed to solid tumours or carcinomas. Where the cancer is present in the prostate, the cancer is termed “prostate cancer”, which is typically characterised by increasing and/or elevated levels of serum prostate-specific antigen (PSA), overexpression of a PSMA membrane protein. A subject may have prostate cancer, where the cancer is a primary cancer and localised in the prostate gland. The prostate cancer may metastasise and spread to other parts of the subject. A subject may also have recurrent prostate cancer, which is characterised by an increase in PSA within 10 years of primary prostate cancer treatment.

Other types of GRPR-expressing cancers that may be treated or imaged by administration of ⁶⁴Cu/⁶⁷Cu-Sar-BBN include prostate cancer, breast cancer, glioma, ovarian cancer, lung cancer, and gastrinoma and gastrointestinal stromal tumours [GIST]).

There are various forms of prostate cancer, each characterised by the location of the cancer, the cell types involved in the cancer (i.e. histology) and levels of prostate specific antigen (PSA) in the patient. Prostate cancer may also be described by the stage and/or grade of the cancer, for example, according to the Gleason score, the size and/or location of the tumor, the presence of one or more tumors in lymph nodes and the degree of metastasis. One form of prostate cancer includes metastatic castrate resistant prostate cancer (mCRPC). Documented progressive mCRPC is based on at least 1 or more of the following criteria:
a. serum/plasma prostate specific antigen (PSA) progression defined as 2 consecutive increases in PSA over a previous reference value measured at least 1 week prior. The minimal value being 2.0 ng/mL;
b. Soft-tissue progression defined as a ≥20% increase in the sum of the diameter (SOD) (short axis for nodal lesions and long axis for non-nodal lesions) of all target lesions based on the smallest SOD since the last treatment directed at the metastatic cancer has started (not including hormonal therapy) or the appearance of 1 or more new lesions; and
c. Progression of bone disease: evaluable disease or new bone lesions(s) by bone scan;
d. That they express GRPR (positive ⁶⁴Cu-SAR-BBN scan) and one of the criteria above to define it as GRPR-positive mCRPC.

There are also various forms of breast cancer, characterised according to whether the cancer is invasive or non-invasive, the location of the cancer and the presence/sensitivity of certain receptors on the cancer cell. For example, non-invasive breast cancers have abnormal cells that are contained within the milk ducts (ductal) or lobules (lobular) of the breast and has not spread into surrounding tissues, whereas invasive forms of the same cancers has spread into surrounding tissue. The type of breast cancer may be classified according to the receptors present on the cancer cells. For example, hormone-receptor positive breast cancers show sensitivity to oestrogen and/or progesterone, while HER2-positive breast cancers show an increase in human epidermal growth factor receptor 2 (HER2) on the surface of the cells. Breast cancer may also be classified according to whether the cancer is contained or has spread to the lymph nodes or more distant parts of the body.

The methods disclosed herein for the treatment of a cancer include the treatment of GRPR-expressing breast cancer, which may be further characterised in accordance with the subtypes discussed above.

The term “patient” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a male human.

The term “therapeutically effective amount” or “effective amount” is an amount sufficient to effect beneficial or desired clinical results. An effective amount can be administered in one or more administrations. For the purposes of radioimaging, an effective amount is sufficient for an image showing the localisation of the compound of Formula (I) administered to the subject, owing to the detection of the products of decay from the radioisotope that is complexed with the compound. For the purposes of treatment, an effective amount is typically sufficient to palliate, ameliorate, stabilize, reverse, slow and/or delay the progression of the cancer.

Radiological progression free survival (rPFS) is defined as the time from first ⁶⁷Cu-SAR-BBN treatment date to radiographic progression on bone scan or radiographic soft tissue progression or death from any cause, whichever comes is first. rPFS will be presented with a Kaplan-Meier curve and summary statistics (median and rPFS at 6, 9, and 12 months). In certain embodiments the method provides a rPFS of from over 6 months to over 5 years.

In some embodiments the effective amount is an amount that is effective to elicit one of the following:
Complete Response (CR): Disappearance of all target lesions. All pathological lymph nodes must have decreased to <10 mm in short axis. Disappearance of all target lesions. Any pathological lymph nodes (whether target or non-target) must have reduction in short axis to <10 mm;
Partial Response (PR): At least a 30% decrease in the sum of diameters of target lesions, taking as reference the baseline sum diameters.

The treatment regime will typically involve a number of cycles (e.g. 1, 2, 3, 4, 5, or 6) of treatment with the cycles being continued until such time as the condition has been ameliorated. Once again the optimal number of cycles and the spacing between each treatment cycle will depend upon a number of factors such as the height and weight of the subject, the severity of the condition being treated, the health (or lack thereof) of the subject being treated and their previous reactions to radiotherapy and/or the extent of the condition as determined through radioimaging.

The formulations defined in the present specification for methods of treatment may be administered parenterally, with intravenous administration preferred. In an embodiment, the aqueous formulation comprising a radiolabelled compound of Formula (I) is administered intravenously, either by bolus administration or infusion.

It will be understood that the specific dose of the radiolabelled compound of Formula (I) for any particular subject will depend upon a variety of factors including, for example, the age, body weight and indication of the individual to be treated, the time of administration, rate of excretion, and combination with any other treatment or therapy. Single or multiple administrations can be carried out with dose levels and pattern being selected by the treating physician. Dosage regimens may be adjusted to provide the optimum therapeutic response. For example, a given dose delivering a certain amount of radiation may be calculated as a fraction of the total radiation to be delivered to the subject. Dosage regimens may comprise the administration of multiple doses of the radiolabelled compound of Formula (I), where the doses are the same or different. In some embodiments, the methods for treatment of a prostate cancer as described herein may comprise the administration of multiple doses of a compound of Formula (I) complexed with a copper-67 radioisotope, where the doses are the same. In other embodiments, the methods comprise the administration of multiple doses, where the second and subsequent doses are higher than the first dose administered to the subject. For instance the first dose may deliver a first does level of about 6 GBq and a second subsequent does may deliver a dose of about 10 GBq. In some embodiments, the dose administered for the purpose of treatment or therapy is determined by prior radioimaging of the subject by administration of the compound of Formula (I) complexed with a ⁶⁴Cu radioisotope in order to locate the cancer sites, estimate the amount of the compound retained by the subject (and subsequently the amount of radioactivity delivered) and assess the nature of the cancer sites. The present inventors believe that the use of the compound of Formula (I) or a pharmaceutically acceptable salt thereof for both radioimaging and radiotherapy represents a theranostic approach to GRPR expressing metastatic castrate resistance prostate cancer.

That is, the present methods for radioimaging and treatment of a GRPR expressing metastatic castrate resistance prostate cancer where the compound of Formula (I) has a different isotope administered, the methods disclosed herein represent a theranostic approach, i.e. a therapeutic and diagnostic (identification) approach, to the treatment of such cancers. This is because administration of the compound of Formula (I) may also be complexed with a radioisotope that allows for the radioimaging of a subject, while administration of the compound of Formula (I) that is complexed with ⁶⁷Cu allows for treatment of the subject. Radioimaging allows for visualisation (identification) of where the compound of Formula (I) accumulates, which then corresponds to the site of treatment. Without wishing to be bound by theory, the present inventors believe that the methods and uses disclosed herein allow for more efficacious treatment of GRPR expressing metastatic castrate resistance prostate cancer. The use of a compound of Formula (I) or a pharmaceutically acceptable salt thereof in combination with a copper radioisotope allows for a higher dose of radioactivity to be delivered in a single dose. Since the compound of Formula (I) is specific for the GRPR membrane protein and retains the copper radioisotope for a longer time (when compared to other metal chelators), the radioactivity is delivered to the cancer site and localised more efficiently. The compound of Formula (I) also shows better clearance from key organs. This in turn reduces any off-target effects of the radioisotope and limits unwanted damage to healthy tissue attributed to dissociation and subsequent circulation of the radioisotope. Better clearance of the radiolabelled compound of Formula (I) and retention at the targeted cancer site leads to images of higher contrast and subsequently more reliable diagnostic images. The ability to deliver a more sustained radiation dose by administration of the compound of Formula (I) complexed with a copper radioisotope also leads to more efficient treatment overall, as smaller amounts of the compound of Formula (I) and the radioisotope are required. Where the requisite radiation is delivered in fewer doses, this results in better tolerance to treatment by the subject.

The present invention also contemplates combination therapies, wherein the radiolabelled compound of Formula (I) as described herein is co-administered with other suitable agents that may facilitate the desired therapeutic outcome. The term “co-administered” means simultaneous administration in the same formulation or in two different formulations via the same or different routes or sequential administration by the same or different routes. The term “concomitant” refers to the administration of more than one formulation, where the formulations are administered to the subject at the same time. The term “simultaneously” means that the active agents are administered at substantially the same time. The term “sequential” administration means a time difference of from seconds, minutes, hours or days between administrations of the agents. Administration may be in any order.

Since the methods disclosed herein relate to the administration of a radioisotope that emits ionising radiation, co-administration of one or more amino acids with the aqueous formulations comprising a radiolabelled compound of Formula (I) as disclosed herein may prevent or limit nephrotoxicity resulting from retention of the radiopharmaceutical. The one or more amino acids co-administered to a subject undergoing treatment for a cancer associated with overexpression of a GRPR membrane protein competitively inhibit the reabsorption of the radiolabelled compound of Formula (I) by the proximal tubules of the kidneys. The present inventors believe that limiting reuptake of the radiolabelled compound of Formula (I) and therefore reducing nephrotoxicity of the subject allows for higher doses of the compound to be administered and therefore increase the efficiency of treatment. The methods for the treatment of a cancer as disclosed herein further comprise the administration of one or more amino acids, or salts thereof, to the subject. In one embodiment, a formulation comprising one or more amino acids or salts thereof is co-administered with an aqueous formulation comprising a compound of Formula (I) complexed with a ⁶⁷Cu radioisotope. In one embodiment, the one or more amino acids includes lysine or a salt thereof. In another embodiment, the one or more amino acids includes arginine or a salt thereof. In a preferred embodiment, the method for the treatment of a cancer further comprises the administration of lysine and/or arginine, or salts thereof. In a preferred embodiment, the method for the treatment of a cancer further comprises the administration of lysine and arginine or salts thereof. In a preferred embodiment, the method for the treatment of a cancer as disclosed herein further comprises the co-administration of lysine and arginine or salts thereof with the compound of Formula (I) complexed with a ⁶⁷Cu radioisotope. In some embodiments, the one or more amino acids, or salts thereof, are administered as an intravenous infusion. In some embodiments, the formulation comprising one or more amino acids comprises L-lysine or a salt thereof. In other embodiments, the formulation comprising one or more amino acids comprises L-arginine or a salt thereof. In some embodiments, the one or more amino acids are present as a hydrochloride salt. In some embodiments, the one or more amino acids are each present at a concentration of about 2.5% w/v.

The methods of the present invention may further comprise the administration of an anti-emetic agent. In one embodiment, the methods of the present invention further comprises the administration of an anti-emetic agent to the subject. In some embodiments, the anti-emetic is administered concurrently with or prior to the compound of Formula (I) complexed with ⁶⁷Cu.

The methods of treatment as disclosed herein comprise the administration of a formulation comprising a compound of Formula (I) complexed with ⁶⁷Cu. The formulation may be administered intravenously, for example, by slow intravenous infusion. The methods of treatment as disclosed herein may comprise a single administration of the formulation comprising a compound of Formula (I) complexed with ⁶⁷Cu, or more than one administration of the same or different formulation. In an embodiment, the method for the treatment of a cancer comprises the administration of one dose of an aqueous formulation comprising a compound of Formula (I) complexed with ⁶⁷Cu. In another embodiment, the method comprises the administration of two doses of an aqueous formulation comprising a compound of Formula (I) complexed with ⁶⁷Cu. In another embodiment, the method comprises the administration of three doses of an aqueous formulation comprising a compound of Formula (I) complexed with ⁶⁷Cu. In another embodiment, the method comprises the administration of four doses of an aqueous formulation comprising a compound of Formula (I) complexed with ⁶⁷Cu. Where four doses are administered the maximum planned cumulative administered activity of ⁶⁷Cu-SAR-BBN will not exceed the critical organ dose limits (23 Gy to the kidney and 2 Gy to the bone marrow) across the 4 administrations.

Where more than one administration is required, the interval between doses of the formulation may be between about 6 weeks to about 14 weeks. In an embodiment, the method comprises the administration of more than one dose of an aqueous formulation comprising a compound of Formula (I) complexed with ⁶⁷Cu, where the doses are administered about 6 weeks apart. In another embodiment, the method comprises the administration of more than one dose, where the doses are administered about 8 weeks apart. In another embodiment, the method comprises the administration of more than one dose, where the doses are administered about 10 weeks apart. In another embodiment, the method comprises the administration of more than one dose, where the doses are administered about 12 weeks apart. In another embodiment, the method comprises the administration of more than one dose, where the doses are administered about 14 weeks apart. In some embodiments, the methods for treatment discussed herein comprise the administration of two or more doses of a formulation comprising a compound of Formula (I) complexed with ⁶⁷Cu, where the time between the doses may be the same. In some embodiments, the method comprises the administration of two or more doses of the formulation, where the time between doses is about the same, for example, about 6 weeks between each dose, about 8 weeks between each dose, about 10 weeks between each dose, about 12 weeks between each dose or about 14 weeks between each dose. In other embodiments, the time between doses may be different, for example, about 6 weeks between the first and second doses, and about 8 weeks between the second and third doses. Other embodiments where the time between different doses are also contemplated, where the time between two sequential doses may be about 4 weeks, about 6 weeks, about 8 weeks, about 10 weeks, about 12 weeks, about 14 weeks or about 16 weeks.

In an embodiment, the method for the treatment of GRPR expressing metastatic castrate resistance prostate cancer comprises the administration of one dose of a formulation comprising a compound of Formula (I) complexed with ⁶⁷Cu. In some embodiments where the formulation comprising the compound of Formula (I) complexed with ⁶⁷Cu is administered more than once, the formulation that is administered on each occasion is the same or different. Where the formulation is different, the compound of Formula (I) complexed with ⁶⁷Cu in each formulation may deliver different doses of radioactivity, for example, about 6 GBq, about 10 GBq, about 14 GBq, about 18 GBq, about 22 GBq or about 24 GBq. In an embodiment, the aqueous formulation of a compound of Formula (I) complexed with ⁶⁷Cu that is administered for a method for the treatment of a cancer associated with overexpression of a GRPR membrane antigen delivers a dose of radiation between about 6 GBq to about 24 GBq to the subject. In an embodiment, the aqueous formulation of a compound of Formula (I) complexed with ⁶⁷Cu that is administered for a method for the treatment of a cancer associated with overexpression of a GRPR membrane antigen delivers a dose of radiation between about 6 GBq to about 14 GBq to the subject. In an embodiment, the aqueous formulation delivers a dose of about 4 GBq. In another embodiment, the aqueous formulation delivers a dose of about 6 GBq. In another embodiment, the aqueous formulation delivers a dose of about 8 GBq. In another embodiment, the aqueous formulation delivers a dose of about 9 GBq. In another embodiment, the aqueous formulation delivers a dose of about 10 GBq. In another embodiment, the aqueous formulation delivers a dose of about 12 GBq. In yet another embodiment, the aqueous formulation delivers a dose of about 14 GBq. In another embodiment, the aqueous formulation delivers a dose of about 16 GBq. In another embodiment, the aqueous formulation delivers a dose of about 18 GBq. In another embodiment, the aqueous formulation delivers a dose of about 20 GBq. In another embodiment, the aqueous formulation delivers a dose of about 22 GBq. In another embodiment, the aqueous formulation delivers a dose of about 24 GBq.

Alternatively, the dose of radioactivity that is delivered by the compound of Formula (I) complexed with ⁶⁷Cu is the maximum dose that is tolerated by the individual subject. One skilled in the art would understand that the maximum tolerated dose will vary among subjects. The present inventors have found that the administration of a compound of Formula (I) with a radioisotope that is suitable for imaging may allow not only for the distribution and uptake of the radiolabelled compound to be visualised, but also the tolerance the subject has for the given dose. Where the subject appears to tolerate the dose well and other physiological measures (e.g. liver and kidney function) are satisfactory, this information may be used to determine a higher dose of radiation that is specific to the subject. In an embodiment, the methods disclosed herein include the step of evaluating the tolerance of a subject to a dose of the compound of Formula (I) complexed with ⁶⁷Cu and modifying the dose of radioactivity that is delivered to the subject in a subsequent dose. Those skilled in the art will understand that a variety of techniques, including nuclear imaging, comparison between baseline levels of radiation and levels after administration of the compound of Formula (I) complexed with ⁶⁷Cu to determine uptake, comparison between of the size and number of lesions before and after administration and monitoring of biochemical markers through one or more diagnostic assays of tissues may be used.

The present invention therefore also provides a method for the treatment of a GRPR expressing metastatic castrate resistance prostate cancer, where the dose of the compound of Formula (I) complexed with ⁶⁷Cu that is administered to the subject is specific to the subject and determined through a combination of imaging and physiological assay techniques. In an embodiment, the dose is determined by the administration of a compound of Formula (I) complexed with a suitable radioisotope to the subject and followed by imaging of the subject for a time to obtain one or more images that can be used to determine the suitability of the administered dose for the subject. In some embodiments, the imaging may be performed by one or more techniques, such as PET, SPECT, CT and MRI. Without wishing to be bound by theory, the present inventors believe that the methods disclosed herein allow for more sophisticated and personalised regimes for the treatment of a cancer in a patient with GRPR expressing metastatic castrate resistance prostate cancer as well as a patient diagnosed with GRPR expressing metastatic castrate resistance prostate cancer who is ineligible for therapy with ¹⁷⁷Lu-PSMA-617.

In a further aspect, the present invention provides a method for predicting the response of a patient to treatment of a cancer with a compound of Formula (I) complexed with ⁶⁷Cu, the method comprising detecting and enumerating circulating tumor DNA (ctDNA) associated with one or more genes in the patient and correlating the amount of ctDNA detected with the response of the patient to said treatment, wherein the cancer is a GRPR expressing cancer and wherein the one or more genes is selected from the group consisting of BRCA1, BRCA2, RB and p53.

In an embodiment, the GRPR expressing cancer is selected from prostate cancer, breast cancer, glioma, ovarian cancer, lung cancer, and gastrinoma and gastrointestinal stromal tumours [GIST]).

As used herein, the term “circulating tumor DNA” (ctDNA) refers to fragments of DNA in the blood of cancer patients released from cancerous cells (e.g. tumor cells) of said patients via apoptosis, necrosis or active release.

In accordance with the method for predicting the response of a patient to treatment of a cancer with a compound of Formula (I) complexed with ⁶⁷Cu and as disclosed herein, ctDNA of a patient is obtained and quantified by appropriate means. The nature and the quantity of the ctDNA that is analysed will depend on the patient and the nature of the cancer. Once determined, a comparison of the nature and quantity of the ctDNA with benchmark values can predict whether a patient may (or may not) respond to treatment with the compound of Formula (I) complexed with ⁶⁷Cu.

In certain embodiments, the ctDNA that is analysed is associated with BRCA1. In other embodiments, the ctDNA that is analysed is associated with BRCA2. In other embodiments, the ctDNA that is analysed is associated with RB. In other embodiments, the ctDNA that is analysed is associated with p53.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### EXAMPLES

The following examples are illustrative of the disclosure and should not be construed as limiting in any way the general nature of the disclosure of the description throughout this specification.

### Example 1—Preparation of ⁶⁴Cu-SAR-BBN and ⁶⁷Cu-SAR-BBN

The synthesis of SAR-BBN begins with the solid phase synthesis of a linear 9 amino acid chain (peptide), followed by the on-resin coupling of a PEG spacer and the (Boc)₄₋₅MeCOSar solution phase chelator. A final capping step with Z-L-Ala-OH terminates any peptide chains that have not coupled with (Boc)₄₋₅MeCOSar. The completed peptide is cleaved from the resin and globally deprotected, purified by reverse-phase, high-performance liquid chromatography (RP-HPLC), and salt exchanged to the acetate salt.

### Solid Phase Peptide Synthesis

The amino acids are added sequentially, via a proven chemistry sequence involving: deprotection of the terminal amino group, activation of the carboxyl group of the next amino acid and then washing the resultant peptide before beginning the next step. The peptide (Boc)₄₋₅MeCOSar-(PEG)₄-D-Phe-Gln(Trt)-Trp(Boc)-Ala-Val-Gly-His(Trt)-Sta-Leu-Rink Resin is prepared using a 11-cycle process.

### Resin Cleavage and Global Deprotection

The resin-bound fully-protected SAR-BBN peptide is treated with a trifluoroacetic acid in the presence of a cation scavenger to cleave the peptide from the resin and remove all the acid labile protecting groups, i.e. the Boc groups of the MeCOSar and orthogonal protecting groups of the peptide. The solution of the crude product is then lyophilized using the following sequence: the product solution is pre-frozen (−60 °C) as a shell on the interior surface of round-bottom flasks and placed on the vacuum manifold at ambient temperature. Maximum achievable vacuum (0.1-0.4 mBar) is applied to the lyophilizer until the product is confirmed as dry by manual checking for solid ice, typically after 30 to 48 hours. The crude product, MeCOSar-(PEG)4-D-Phe-Gln-Trp-Ala-Val-Gly-His-Sta-Leu-NH2, is then analysed by RP-HPLC to confirm the quality of the material and by mass spectral analysis to confirm that the product is fully deprotected (removal of all the Boc groups).

### Purification

The crude SAR-BBN is then subjected to preparative RP-HPLC purification as the trifluoroacetate salt. Fractions are collected in tubes from each purification run and analyzed by analytical HPLC; fractions of acceptable purity are combined and lyophilized under the same conditions.

### Acetate Exchange

The trifluoroacetate salt form is exchanged to the acetate salt form by preparative RP-HPLC using ammonium acetate and an eluent system of acetic acid (AcOH) in water/acetonitrile.

### Freeze-Drying of Bulk Material

The bulk SAR-BBN acetate salt is lyophilized under conditions described for the crude SAR-BBN above. The product is weighed into a screw-capped storage bottle, vacuum sealed and stored refrigerated prior to aliquoting into vials in the desired quantity for radiolabelling.

⁶⁴Cu-SAR-BBN was prepared by addition of [64^{Cu}]CuCl₂ (1000-2000 MBq, 100-500 μL, 0.05 M HCl) to SAR-BBN (60 μg) in a 5 mL solution of sodium phosphate buffer (0.1 M, pH 6.5-7.0) containing sodium gentisate (5.7 mg). The reaction mixture was incubated for 25 min at room temperature, after which the reaction mixture was filtered through a 0.22 μm filter into a sterile product vial. The reaction was quenched by addition of 10 mL of a 7.5% aqueous ethanol solution containing sodium ascorbate (1.17 g) via the 0.22 μm filter into the sterile product vial. ⁶⁴Cu-SAR-BBN was produced in an average 84% radiochemical yield with more than 95% radiochemical purity.

⁶⁷Cu-SAR-BBN was prepared by addition of [⁶⁴Cu]CuCl₂ (6000-12000 MBq, 100-500 μL, 0.05 M HCl) to SAR-BBN (120 μg) in a 14 mL solution of sodium phosphate buffer (0.1 M, pH 6.5-7.0) containing sodium gentisate (11.4 mg). The reaction mixture was incubated for 25 min at room temperature, after which the reaction mixture was filtered through a 0.22 μm filter into a sterile product vial. The reaction was quenched by addition of 13 mL of an 8% aqueous ethanol solution containing sodium ascorbate (2.27 g) via the 0.22 μm filter into the sterile product vial. ⁶⁴Cu-SAR-BBN was produced in an average 86% radiochemical yield with more than 95% radiochemical purity.

### Example 2—Non-Clinical Studies

A number of non-clinical studies were conducted to determine the biodistribution, in vivo safety and tolerability as well as tumor imaging and efficacy of SAR-BBN labelled with either natural stable Cu (^{nat}Cu), ⁶⁴Cu or ⁶⁷Cu.

A PCa xenograft mouse model was injected with ⁶⁴Cu-Sar-BBN, which showed high uptake and retention of the radiolabelled compound over a period of 24 hours (19.6% injected activity (IA)/g at 1 hour and 7.9% IA/g at 24 hours post-injection. These results compare favourably to other GRPR targeted ligands in similar pre-clinical models.

Preclinical efficacy data of ⁶⁷Cu-SAR-BBN in mice showed statistically significant tumor growth inhibition and increased survival compared to the control group in a PCa xenograft study.

A repeat dose toxicology study of the compound of Formula (I) was conducted with weekly administration to male and female mice for 4 weeks by IV injection. Abnormal clinical signs in mice administered with ^{nat}Cu-Sar-BBN were only observed on injection days. In male mice treated with 2 mg/kg ^{nat}Cu-Sar-BBN, fully reversible microscopic findings were observed, however these findings were not considered to be adverse and no other clinical abnormalities, significant changes in weight or feed intake, effects in haematology, blood biochemistry or urine analyses were observed. This establishes the no observed adverse event level at 2 mg/kg for ^{nat}Cu-Sar-BBN for both male and female mice.

### Example 3—Biodistribution of ⁶⁷Cu-Sar-BBN

The biodistribution of ⁶⁷Cu-SAR-BBN was investigated in healthy male and female mice. Effective blood clearance of ⁶⁷Cu-SAR-BBN was demonstrated at 1 hour in male (0.79±0.22% IA/g) and female (1.06±0.18% IA/g) mice, which further reduced over time. The expression of GRPR in the pancreas resulted in high initial pancreatic uptake, with the highest accumulated activity at 1 hour in both male and female mice (19.43±9.98% IA/g and 22.32±10.54% IA/g, respectively), with over 75% and 97% of the ⁶⁷Cu-SAR-BBN activity in the pancreas cleared at 4 and 24 hours, respectively. A similar rapid clearance profile was seen in other GRPR-expressing organs, specifically the adrenals and the stomach. ⁶⁷Cu-SAR-BBN showed low uptake in the kidneys in both male and female mice, even at 1 hour (3.79±0.542% IA/g and 6.15±0.73% IA/g, respectively), which further reduced over time, suggesting a fast renal clearance. Uptake in the liver and intestines suggests there is also hepatobiliary clearance of ⁶⁷Cu-SAR-BBN, which deceased consistently over 24-216 hours. Blood data showed rapid clearance of blood pool activity and relatively rapid clearance of renal and hepatic activity.

Full dosimetry calculations from the mice biodistribution study enabled organ radiation dose extrapolations for the adult male for both the therapeutic, ⁶⁷Cu-SAR-BBN, and the diagnostic, ⁶⁴Cu-SAR-BBN to be calculated. For ⁶⁷Cu-SAR-BBN, the highest absorbed radiation dose is estimated to be the liver at 0.109 mGy/MBq, followed by the urinary bladder wall at 0.641 mGy/MBq. Similarly, for ⁶⁴Cu-SAR-BBN, the largest absorbed radiation dose is estimated to be the urinary bladder wall and liver, with values of 0.070 mGy/MBq and 0.040 mGy/MBq, respectively. The whole body effective dose for ⁶⁴Cu-SAR-BBN was estimated to be 0.018 mSv/MBq for adult males.

These results indicate targeted delivery of ⁶⁴Cu-SAR-BBN and ⁶⁷Cu-SAR-BBN to the tumor sites and show the therapeutic efficacy of the radiolabelled compounds in GRPR-expressing cancers in humans.

Blood data showed rapid clearance of blood pool activity and relatively rapid clearance of renal and hepatic activity. These dosimetry results indicate that the red marrow is likely to be the dose-limiting organ for ⁶⁷Cu-SAR-BBN, with an estimated absorbed dose of 0.023 mGy/MBq. The pancreas is estimated to be the organ with the highest absorbed dose (0.303 mGy/MBq), while the absorbed dose for the kidneys is estimated to be 0.070 mGy/MBq. No clear dose limit for radiation therapy for the pancreas is given in the literature, and the pancreas is not yet considered as an organ at risk for radiotherapy planning.

The absorbed doses to be received from the planned administered activities of ⁶⁷Cu-SAR-BBN in the study, were assessed against the 2 Gy bone marrow absorbed dose limit (shown in Table 1). To reach the 2 Gy kidney threshold, it has been estimated that an administered activity of ~87 GBq of ⁶⁷Cu-SAR-BBN would need to be given to an adult male. The proposed dose levels have a total administered activity range of 6-56 GBq, which would result in a total absorbed dose to the bone marrow of 0.138-1.288 Gy, that is well below the established limit at all proposed doses.

### Example 4—Formulations of Sar-BBN

Both ⁶⁴Cu-Sar-BBN and ⁶⁷Cu-Sar-BBN are formulated as sterile solutions for IV injection suitable for human use.

⁶⁴Cu-SAR-BBN and ⁶⁷Cu-SAR-BBN are stored at room temperature in a sealed, sterile, pyrogen-free glass vial with an expiration time designation on the label. The shelf-life of ⁶⁴Cu-SAR-BBN and ⁶⁷Cu-SAR-BBN is indicated on the product label.

### Example 5—Administration of ⁶⁴Cu-Sar-BBN for Radioimaging

A dose of 200 MBq ⁶⁴Cu-Sar-BBN was administered to a subject by slow intravenous bolus injection. The subject was imaged by PET/CT and the images analysed to determine the location, size and volume of any cancerous lesions present, which is indicated by the decay products of the ⁶⁴Cu radioisotope detected.

The images of the subject produced as described above were used to determine the suitability of the subject for treatment of a cancer (where present) with ⁶⁷Cu-Sar-BBN.

### Example 6—Diagnostic Imaging Using Conventional ¹⁸FDG PET or ⁶⁴Cu-Sar-BBN PET in Breast Cancer Patients

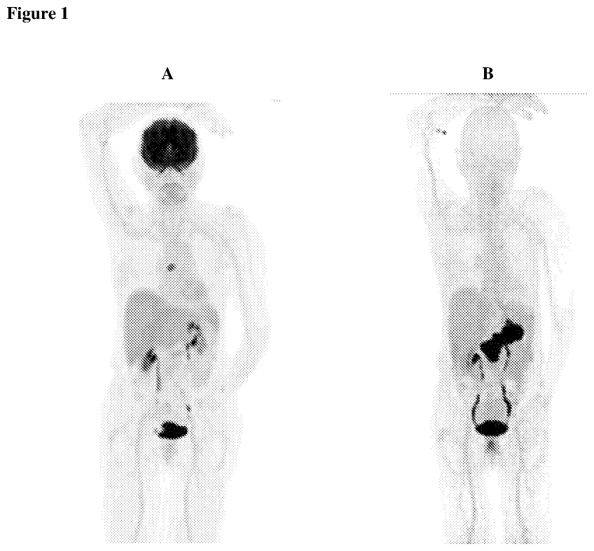

Women with clinical progression of metastatic ER+/PR+/HER2− breast cancer requiring re-staging were administered ¹⁸FDG and images of each patient acquired by PET. Imaging with ⁶⁴Cu-Sar-BBN PET (with 200 MBq ⁶⁴Cu-Sar-BBN administered by IV injection) was performed within 2 weeks of imaging by ¹⁸FDG. Qualitative interpretation of the PET studies was performed by accredited nuclear medicine physicians. Quantitative analysis was performed using MIM Software, Cleveland. Comparison of the images obtained from each subject after administration of both ¹⁸FDG and ⁶⁴Cu-Sar-BBN can be seen in FIGS. 1 to 6 (see Table 1). These figures show that ⁶⁴Cu-Sar-BBN targeting GRPR receptors located lesions in the subjects that were not identified during imaging with other modalities.

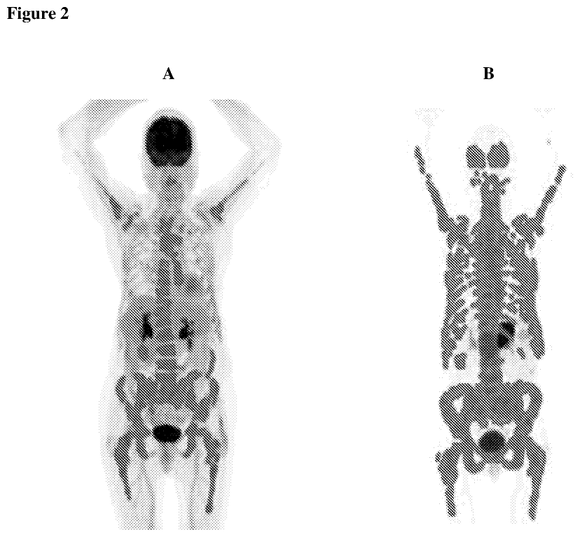

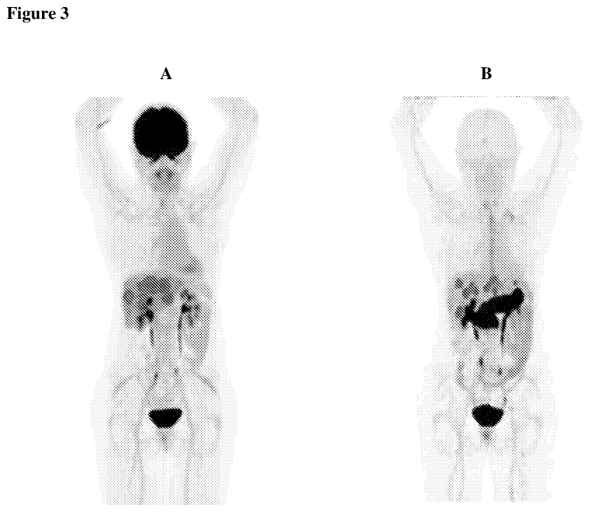

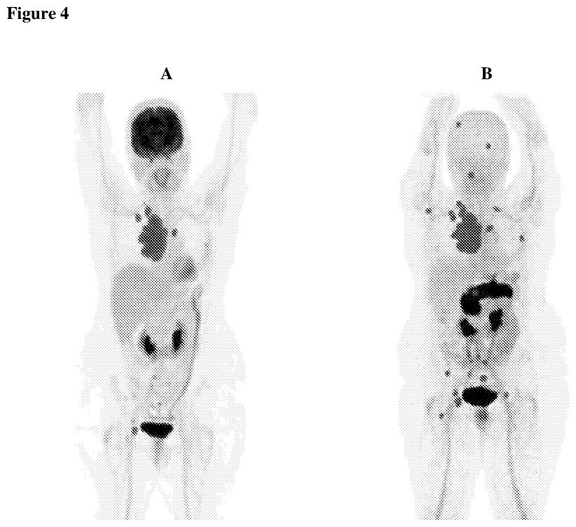

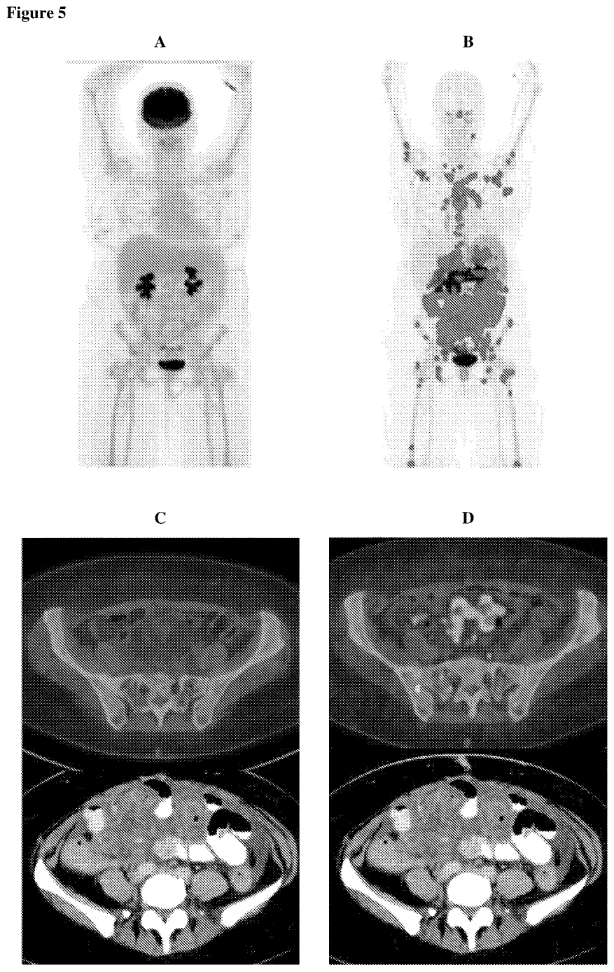

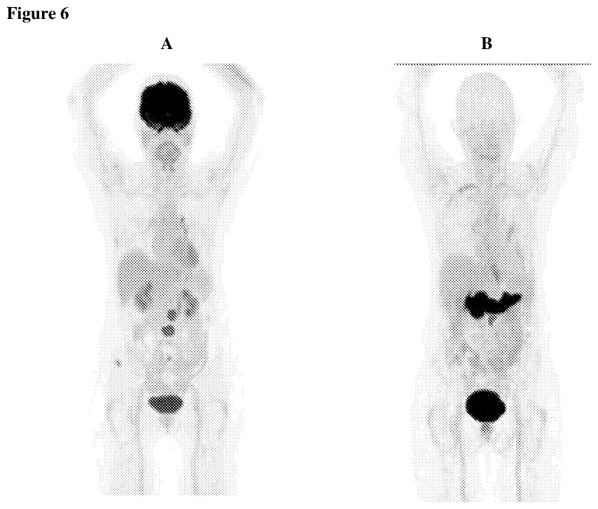

TABLE 1
Comparison of imaging with ¹⁸FDG and
⁶⁴Cu-Sar-BBN in breast cancer patients.

| | Histological | Metastatic | FDG | | BBN (1 hr) | |
|---|---|---|---|---|---|---|
| Patient | subtype | biopsy site | SUVₘₐₓ | TTV | SUVₘₐₓ | TTV |
| 1 (FIG. 1) | Invasive | Liver | 7 | 2 | <3 | 0 |
| | ductal | | | | | |
| 2 (FIG. 2) | Classical | Liver | 11 | 504 | 20 | 2033 |
| | lobular | | | | | |
| 3 (FIG. 3) | Invasive | Liver | 7 | 91 | 6.4 | 13 |
| | ductal | | | | | |
| 4 (FIG. 4) | Invasive | Chest wall | 20 | 168 | 27 | 209 |
| | ductal | | | | | |
| 5 (FIG. 5) | Classical | Skin | <3 | 0 | 19 | 634 |
| | lobular | | | | | |
| 6 (FIG. 6) | Invasive | Pleura | 8 | 8 | 5 | 3 |
| | ductal | | | | | |

### Example 7—Diagnostic Imaging Using Conventional ¹⁸FDG PET or ⁶⁴Cu-Sar-BBN PET in Prostate Cancer Patients

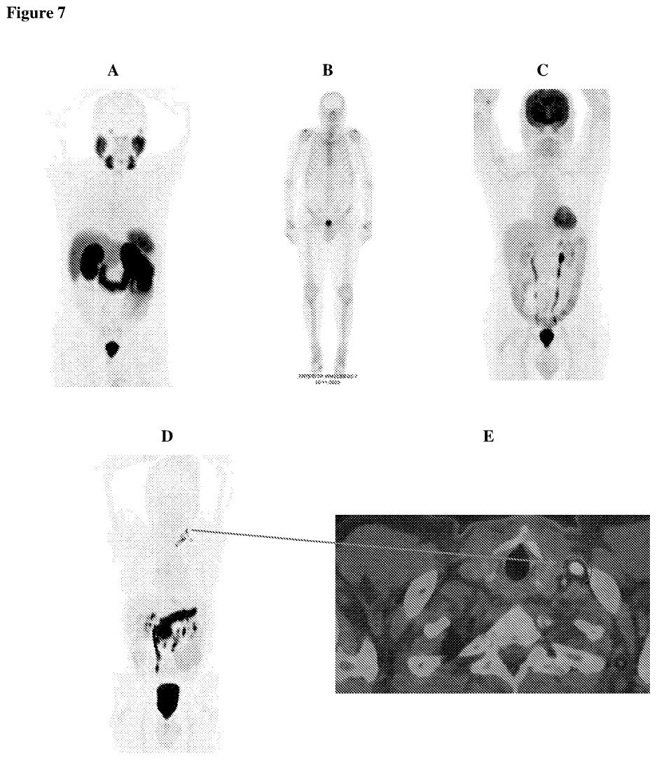

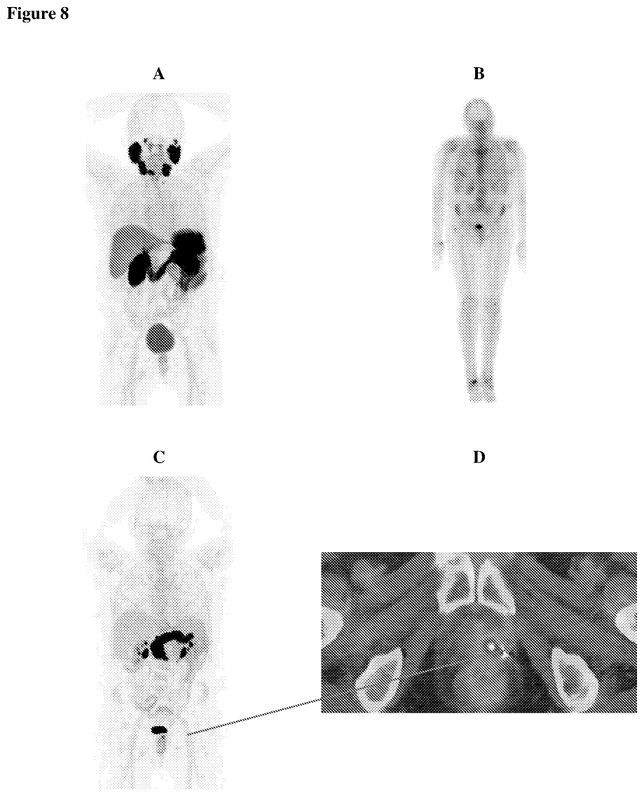

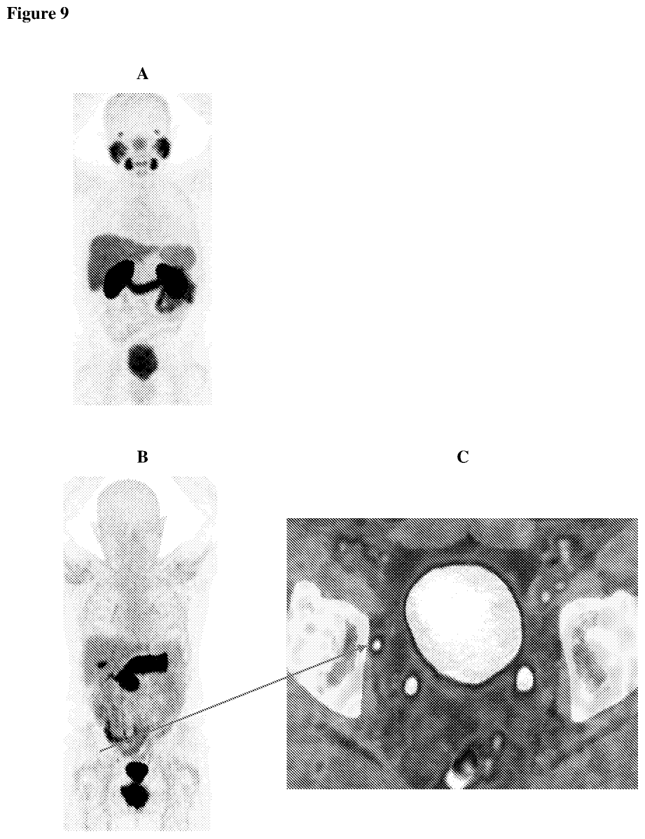

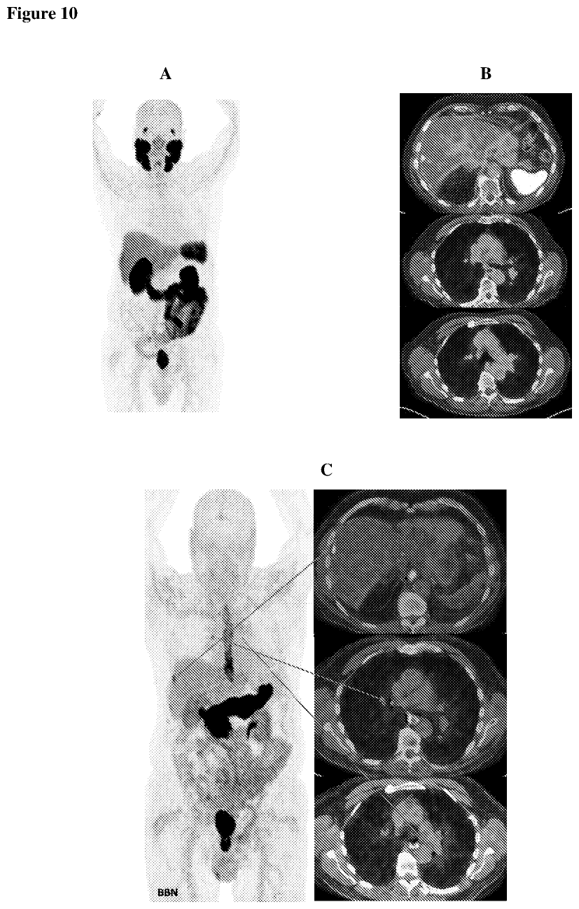

Four men post-radical prostatectomy demonstrated biochemical recurrence with serial negative PSMA-PET (⁶⁸Ga-PSMA-11). Traditionally used imaging modalities including Bone Scan, CT and in some cases, whole body MRI also failed to identify sites of recurrence. All patients received on average 200 MBq of [⁶⁴Cu]Cu-SAR-BBN intravenously with 60 minutes for uptake. Patient were scanned at 60-180 minutes post-injection with arms up, from vertex to mid thighs and at each bed position for two minutes. A low dose CT scan was performed with for attenuation correction and anatomical localisation. Comparison of the images obtained from each subject after administration of both ¹⁸FDG and ⁶⁴Cu-Sar-BBN can be seen in FIGS. 7 to 10. These figures show that ⁶⁴Cu-Sar-BBN targeting GRPR receptors located lesions in the subjects that were not identified during imaging with other modalities.

### Example 8—Qualification of Subjects for Treatment with ⁶⁷Cu-Sar-BBN

Where images of a subject having been administered ⁶⁴Cu-Sar-BBN as obtained by PET/CT show the binding of ⁶⁴Cu-Sar-BBN, the subject is considered to be suitable for treatment with ⁶⁷Cu-Sar-BBN, since ⁶⁴Cu and ⁶⁷Cu are a theranostic pair

### Example 9—Administration of ⁶⁷Sar-BBN for Treatment

The compound of Formula (I) complexed with a ⁶⁷Cu radioisotope (i.e. ⁶⁷Cu-SarBBN) at a dose of 6 GBq was administered to a subject determined to have cancerous lesions associated with GRPR, as identified by radioimaging after administration of ⁶⁴Cu-Sar-BBN.

A further dose of 6 GBq was administered to the same patient at a time of 6 weeks after the initial administration.

After a period of at least two days (i.e. to allow for elimination of 67Cu-Sar-BBN), a further dose of 200 MBq of 64Cu-Sar-BBN was administered to the subject and images of the subject taken by PET/CT. Images taken after the administration of 64Cu-Sar-BBN (both before and after administration of ⁶⁷Cu-Sar-BBN) were used to determine the progression of any lesions present in the subject.

### Example 10—Determining Efficacy of Sar-BBN Efficacy Assessments Tumor Response Assessment

Tumor measurement for disease evaluation was performed with bone scans and CT/MRI, and response was evaluated as per PCWG3 (Prostate Cancer Working Group 3) guidelines. Where patients show evidence of progression by bone scan alone, a confirmatory scan (i.e. by administration of ⁶⁴Cu-Sar-BBN and subsequent imaging) was performed and images further assessed.

### Example 11—Qualitative Analysis of Scans

The ⁶⁴Cu-SAR-BBN PET/CT and baseline standard of care images will be assessed to identify the number of lesions detected. The lesion level analysis compares the number of GRPR-expressing lesions seen on the 64Cu-SAR-BBN PET/CT screening scan with the number of lesions seen on the baseline standard of care images.

Baseline standard of care images to be used for comparison:
CT or MRI scans for soft tissue disease.
Bone scan for the bone lesions.

### Example 12—Detecting and Quantifying ctDNA

Blood was drawn from a patient and analysed using the CELLSEARCH® Circulating Tumor Cell Kit (Janssen Diagnostics, Raritan, NJ). The determined volume/amount of circulating tumour cells (CTCs) or ctDNA is then compared with expected values, for example to a healthy patient or values determined for the patient prior. CTC count will be reported as:
Favourable is 4 or fewer cells per 7.5 mL of blood.
Unfavourable if 5 or more cells per 7.5 mL of blood.

This CTC or ctDNA value is then correlated to the prognosis and response to treatment.

1. A method of treating a patient diagnosed with a GRPR expressing cancer, said method comprising administering an effective amount of a compound of Formula (I) or a pharmaceutically acceptable salt thereof complexed to a ⁶⁷Cu radioisotope: 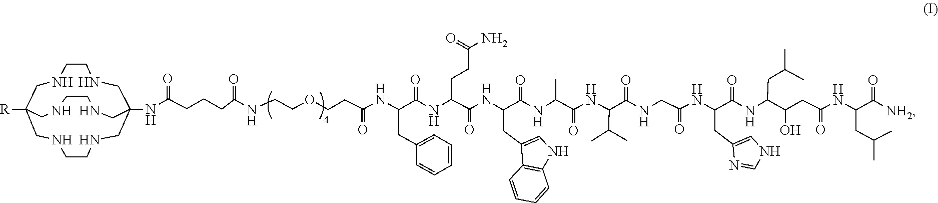
wherein R is CH₃—;
(⁶⁷Cu-SAR-BBN)
and
wherein the dose of radiation delivered by the radioisotope is sufficient to reduce the size of one or more lesions associated with said cancer.

2. The method according to claim 1, wherein the GRPR expressing cancer is selected from the group consisting of prostate cancer, breast cancer, glioma, ovarian cancer, lung cancer, and gastrinoma and gastrointestinal stromal tumours (GIST).

3. The method according to claim 1, wherein the GRPR expressing cancer is a metastatic castrate resistant prostate cancer.

4. The method according to claim 1, wherein the patient is diagnosed with a GRPR expressing metastatic castrate resistant prostate cancer and is ineligible for therapy with ¹⁷⁷Lu-PSMA-617.

5. A method of identifying and treating a patient diagnosed with a GRPR cancer, said method comprising including:
(i) administering an effective amount of a compound of Formula (I) or a pharmaceutically acceptable salt thereof complexed to a ⁶⁴Cu radioisotope: 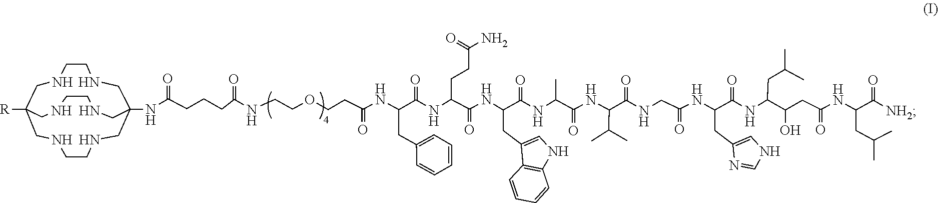
wherein R is CH₃—;
(⁶⁴Cu-SAR-BBN)
and
wherein the dose of radiation delivered by the ⁶⁴Cu radioisotope is sufficient to identify one or more lesions associated with said cancer; and
(ii) administering an effective amount of a compound of Formula (I) or a pharmaceutically acceptable salt thereof complexed to a ⁶⁷Cu radioisotope: 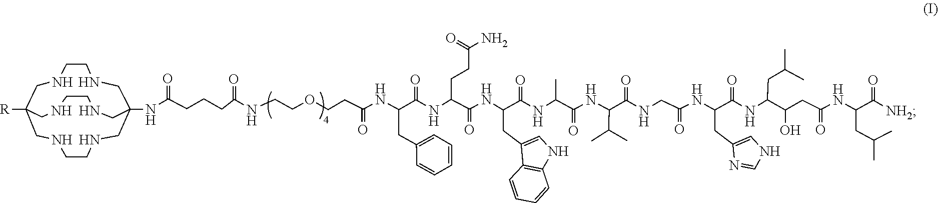
wherein R is CH₃—;
and
wherein the dose of radiation delivered by the ⁶⁷Cu radioisotope is sufficient to reduce the size of said one or more lesions associated with said cancer.

6. The method according to claim 1, wherein the GRPR expressing cancer is GRPR expressing metastatic castrate resistant prostate cancer.

7. The method according to claim 6, wherein the patient is diagnosed with a GRPR expressing metastatic castrate resistant prostate cancer and is ineligible for therapy with ¹⁷⁷Lu-PSMA-617.

8. The method according to claim 1, wherein the stereochemistry of the peptide portion of the compound of Formula (I) is depicted as follows:

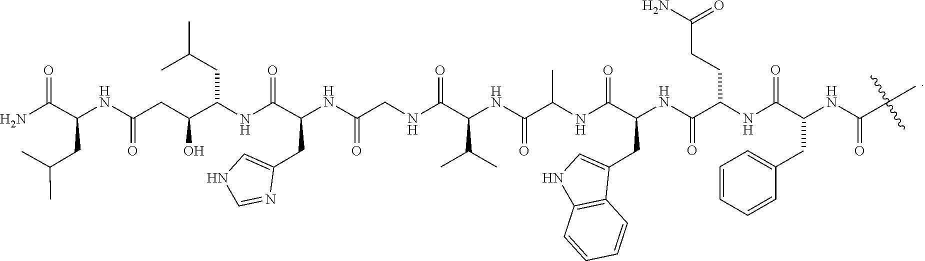

9. The method according to claim 5, wherein the dose of radiation delivered by the ⁶⁴Cu radioisotope is about 200 MBq.

10. The method according to claim 9, wherein the patient is subjected to 1, 2, 3 or 4 administrations of ⁶⁷Cu-SAR-BBN.

11. The method according to claim 10, wherein the maximum planned cumulative administered activity of ⁶⁷Cu-SAR-BBN will not exceed the critical organ dose limits (23 Gy to the kidney and 2 Gy to the bone marrow) across 4 administrations.

12. The method according to claim 1, wherein the patient is a male subject with a castrate level of serum/plasma testosterone of about <50 ng/dL or about <1.7 nmol/L.

13. The method according to claim 1, wherein the GRPR expressing cancer is a GRPR expressing metastatic castrate resistant prostate cancer (GRPRmCRPC), and is a progressive GRPRmCRPC despite prior androgen deprivation therapy and at least either enzalutamide and/or abiraterone (or other such androgen receptor pathway inhibitors).

14. The method according to claim 1, wherein the patient experiences a decrease in percentage of PSA and/or, alkaline phosphatase (ALP) and/or, lactate dehydrogenase (LDA) biomarkers after one, two, three or four treatment administration cycles of a compound of Formula (I) or a pharmaceutically acceptable salt thereof complexed to a ⁶⁷Cu radioisotope relative to a baseline taken prior to each the treatment.

15-16. (canceled)

17. A method for predicting the response of a patient to treatment of a cancer with a compound of Formula (I) complexed with ⁶⁷Cu, the method comprising detecting and enumerating circulating tumor DNA (ctDNA) associated with one or more genes in the patient and correlating the amount of ctDNA detected with the response of the patient to said treatment, wherein the cancer is a GRPR expressing cancer and wherein the one or more genes is selected from the group consisting of BRCA1, BRCA2, RB and p53.

18. (canceled)
